(19) United States
(12) Patent Application Publication
(10) Pub. No.: US 2019/0247205 A1
(43) Pub. Date: Aug. 15, 2019

(54) DEVICE FOR PRODUCING A PLASTER IMPRESSION OF A LIMB STUMP WITH CONNECTOR

(71) Applicant: Andreas RADSPIELER, Neubeuern (DE)

(72) Inventor: Andreas RADSPIELER, Neubeuern (DE)

(21) Appl. No.: 16/338,645

(22) PCT Filed: Oct. 2, 2017
(86) PCT No.: PCT/EP2017/074968

§ 371 Date: Apr. 1, 2019

(30) Foreign Application Priority Data
Oct. 4, 2016 (DE) 10 2016 118 765.8

(51) Int. Cl. A61F 2/50; A61F 2/60

(52) U.S. Cl. CPC A61F 2/5046; A61F 2002/5056; A61F 2002/5052; A61F 2/60

(57) ABSTRACT

A medical apparatus is provided for use in manufacturing a plaster impression of limb stump, in particular of a lower stump, wherein the apparatus comprises a pressure vessel with a fluid container of pressure chamber for receiving or storing a fluid being under pressure, wherein the pressure vessel comprises a wall made of a first material, wherein the wall limits the an interior of of the pressure vessel against an exterior, wherein the pressure vessel comprises an insertion opening for the insertion of a limb stump into the interior of the pressure vessel and a fluid-tight membrane made of a second materials, which is arranged to form or limit the fluid container or the pressure chamber. An upper section of the membrane is preferably releasably fastened at or to the pressure vessel by a connector.

The present invention relates to an apparatus according to the preamble of claim 1.

Leg amputees may regain mobility using leg prostheses. Modern leg prostheses include various modules to which the prosthesis shaft belongs.

The present invention relates to the technical field of producing a plaster impression as a copy, template or model for the later prosthesis shaft of a prosthesis, preferably for the lower extremities, i.e. for a leg prosthesis, in particular a lower leg prosthesis.

An object of the present invention may be to provide an apparatus for use in making a plaster impression for a prosthesis shaft, or at least for an outer shaft thereof, in particular for the lower extremity

The object according to the present invention may be achieved by an apparatus with the features of claim 1.

A medical apparatus (in short: apparatus) is thus provided by the present invention which apparatus may be used in the production of a plaster impression of a limb stump, in particular a lower leg stump. This may be used for example as a basis for producing a lower leg prosthesis shaft, on the, only or merely preferably standing, patient.

In this, the apparatus comprises a fluid container or a pressure vessel with exactly or at least one fluid container. This may receive or store an optionally pressurized fluid. Thereby, the pressure is above the atmospheric pressure. The fluid is a gas or a liquid, preferably air or water, since the latter two are respectively cheap and easily available.

The pressure vessel comprises a wall, which is made of at least or exactly one first material, or comprises at least one first material.

The wall of the pressure vessel limits or delimits or surrounds its interior. According to the present invention, the interior of the pressure vessel is understood to be the space or volume defined by the geometry of the pressure vessel or encompassed or circumscribed by an outer wall of the pressure vessel. If the pressure vessel is for example cylindrical, the interior of the pressure vessel is the space delimited by the cylindrical shell surface and by the two end sides or end planes. If the pressure vessel is, in another example, rectangular, the space of the interior is defined by the result of the multiplication of the height, width and depth of the rectangle. In determining the interior, it is irrelevant whether or not the space corresponding to the interior is fluid-tight. The interior does not represent or is not a fluid-tight closed space but rather a volume circumscribed by the wall. The space which does not belong to the interior of the pressure vessel is referred to herein as its exterior.

The pressure vessel comprises an insertion opening through which the limb stump (which may in this respect, herein also be understood as the distal end of the stump instead of the whole stump) may be inserted into the interior of the pressure vessel. The insertion opening may, for example, be an open end side or end plane, a passage through-opening in the wall or an opening which breaks through or interrupts the wall. In the region of the insertion opening, the interior of the pressure vessel is thus not separated from the exterior by a section of the wall. The insertion opening may lie in an insertion opening or in an insertion plane, through which the limb stump is inserted into the interior of the pressure vessel.

Furthermore, the pressure vessel comprises at least one or exactly one fluid-tight membrane. Alternatively, the pressure vessel does not have such a membrane as described further below, but only correspondingly suitable and/or provided receiving devices (such as the connectors described in the following as optional) for receiving the membrane on/at the pressure vessel.

The membrane is made of or comprises a second material. The first and the second material differ from each other.

At least one section of the membrane, which lies in an upper and/or in a proximal region and/or which faces the insertion opening of the pressure vessel, is connected to at least one connector, preferably releasably. With or by this at least one connector, the membrane is, preferably releasably, fixed or secured or fastened to or at the pressure vessel.

The present invention also relates to a medical apparatus for use in the manufacturing of a plaster impression or a data model of a limb stump, in particular of a lower leg stump, wherein the apparatus comprises a fluid container (may be a pressure vessel) for a liquid, e.g. water. The fluid container comprises, during use of the apparatus, an upper, or first, end side and, during use, a lower, or second, end side. In the region of the second end side, the fluid container can be fluid-tightly closed, for example by a bottom region. The apparatus comprises a film or membrane for receiving a section of a limb stump. The membrane is fluid-tightly connected to the container and/or closes the latter at least at the top or at least at the first end side, at least in a section, fluid-tightly. The fluid container may optionally comprise a pressure chamber. The fluid container comprises optionally at least one fluid outlet, which is optionally provided with a valve or a stopcock for opening and closing the outlet. The membrane is preferably bag-like (i.e. with a dead end like a bag, as opposed to a tube open at the top and at the bottom). At least one upper and/or proximal section and/or a section, which lies in the region which faces the insertion opening of the pressure vessel is fastened directly or indirectly, preferably releasably, by at least one connector at or to a section of a wall of the fluid container, for example a bottom plane or side plane thereof. The membrane is preferably made of, or comprises, a material which has no (or only little) elasticity, or preferably no (or only little) extensibility, at least in a first direction of the material, preferably in a longitudinal direction of the container or in an insertion direction of the limb stump. Instead of having the aforementioned membrane and/or the connector, the apparatus may be designed or configured to be connected to a membrane and/or to a connector. Suitable connecting devices may be provided e.g. at a bottom plane or side wall.

Embodiments according to the present invention of each of the aforementioned subject-matter may comprise one or several of the following features in any combination unless the person skilled in the art recognizes a concrete combination as technically impossible. The subject-matters of the dependent claims also indicate embodiments according to the present invention.

In all of the statements mentioned above and/or below, the use of the expression “may be” and “may have” etc. is to be understood respectively synonymous to “is preferably” or “has preferably,” etc., and is intended to illustrate embodiments according to the present invention.

Whenever numerical words are mentioned herein, the person skilled in the art shall recognize or understand them as indications of numerical lower limits. Hence, unless it leads to a contradiction evident for the person skilled in the art, the person skilled in the art shall comprehend for example “one” as encompassing “at least one”. This understanding is also equally encompassed by the present invention as the interpretation that a numerical word, for example, “one” may alternatively mean “exactly one”, wherever this is evidently technically possible in the view of the person skilled in the art. Both of these understandings are encompassed by the present invention and apply herein to all used numerical words.

In case of doubt, the person skilled in the art will understand the spatial information like “top”, “bottom”, “upper” or “lower”, whenever they are mentioned herein, as a spatial indication with reference to the alignment in the figures appended hereto and/or of the arrangement of the apparatus(es) according to the present invention when used as intended.

The membrane may optionally have several segments or sections. For example, a distal section, in particular a section to which low forces are applied, in particular lower forces compared to other sections of the membrane, may have lower wall thicknesses relative to the one or the other sections. A section of the membrane on which lower forces act, may comprise another, for example softer, material relative to other sections of the membrane. The forces may be tensile forces and/or compressive forces. A further proximal section of the membrane may be made of, or comprise, a second material, wherein the proximal section may comprise a higher wall thickness and/or other material properties relative to the distal section. In particular, the proximal section may be made of, or comprise, composite material. The distal section may be coated on one or both sides.

Several sections of the membrane may be connected to each other cohesively or in material connection, respectively. Alternatively or additionally, the sections of the membrane may be mechanically connected, for example by adapters, clamps, clamping rings or the like.

In certain exemplary embodiments according to the present invention, the pressure chamber is a locked and/or lockable space in which the fluid may be subjected to pressure above the atmospheric pressure (in short: atmosphere) without being able to escape from this space.

In some exemplary embodiments according to the present invention, the membrane is arranged to form the pressure chamber or alternatively to delimit it, e.g., by being part, in particular by being elastic or only in one direction elastic part, of the wall or limitation of the pressure chamber.

In certain exemplary embodiments according to the present invention, the membrane serves for building, by itself (e.g. a balloon) or alternatively together with sections of the wall, a fluid-tight fluid container of the pressure vessel which chamber lies at least partially or completely in the interior of the pressure vessel. Since it can receive and/or hold fluid under a pressure which is above atmospheric pressure, this fluid container is designated herein as pressure chamber.

The terms “fluid container” and “pressure chamber” are interchangeable in certain exemplary embodiments according to the present invention and/or in those embodiments in which the person skilled in the art does not object thereto. What is said herein about the “pressure chamber” may also be applicable to a “fluid container”.

In some exemplary embodiments according to the present invention, the pressure chamber then lies inside the pressure vessel when the pressure prevailing in the pressure chamber does not exceed a certain pressure. Deviating from this, in some embodiments according to the present invention, the pressure chamber extends also to the exterior of the pressure vessel when the pressure prevailing in the pressure chamber pressurizes the membrane such that the latter projects outwards, for example through the insertion opening, i.e. into the exterior of the pressure vessel. The pressure chamber may thus have a variable volume, which depends on the pressure prevailing in the pressure chamber. The latter does not apply to the constant interior or inner volume of the pressure vessel.

In some exemplary embodiments according to the present invention, a fluid may be maintained in the pressure chamber under a pressure above the atmosphere, regardless of the insertion opening interrupting the wall.

In certain exemplary embodiments according to the present invention, the pressure chamber serves for receiving or supporting the distal end of the patient's limb stump which is inserted into the interior of the pressure vessel. Due to the fluid contained in the pressure chamber, the membrane nestles laterally or circumferentially to the distal end of the limb stump or to the entire limb stump. In this way, it may be possible to pressurize the limb stump in the region of the entire plaster bandage through the membrane with the—referring to a region unit—preferably unchanged or same pressure. The latter may be an advantageous contribution of the present invention, since the even pressurization may lead to an even modeling of the moist plaster bandage. Placing the membrane under the fluid stored in the pressure chamber may advantageously make manual modeling unnecessary or significantly reduce the necessary effort. The plaster bandage may be standard.

In some exemplary embodiments according to the present invention, the pressure chamber is a fluid-tight closed space which is entirely, or among others, formed or limited by the wall of the pressure vessel and the membrane. In some exemplary embodiments according to the present invention, the term “pressure chamber” may be replaced by the definition above.

In some exemplary embodiments according to the present invention, the pressure vessel is the vessel or space in which the pressure chamber is arranged.

In some exemplary embodiments according to the present invention, the pressure vessel is a water vessel.

In so some exemplary embodiments according to the present invention, the term “pressure vessel” may be replaced by the term “fluid vessel” or “water vessel”.

In specific exemplary embodiments according to the present invention, the pressure vessel has a cylindrical form.

In some exemplary embodiments according to the present invention, the pressure chamber is designed and/or arranged in such a way that the pressure prevailing therein depends, inter alia or exclusively, on the insertion depth of the limb stump into the interior of the pressure vessel, in any case during the intended use of the apparatus and with closed inlets and outlets, if present.

In certain exemplary embodiments according to the present invention, the membrane, for example a second section of the membrane, in particular a section having an increased strength (e.g. by an increased wall thickness and/or other material properties due to composites, coating or the like) compared to the other section or other sections of the membrane, is arranged in the region of the insertion opening and is optionally there directly or indirectly connected to the wall in a releasable or non-releasable manner, preferably fluid-tight. It closes the insertion opening preferably similar to a cap, insofar the pressure present in the pressure chamber does not deviate significantly from the atmospheric pressure.

In some exemplary embodiments according to the present invention, the membrane is arranged to prevent a fluid or material exchange in the interior of the pressure vessel in its axial direction.

In certain exemplary embodiments according to the present invention, the membrane is always arranged in a single layer in axial and/or radial direction.

In some exemplary embodiments according to the present invention, the membrane is directly or indirectly connected to the pressure vessel on a first end side of it, but not also to a second end side lying opposite the first end side, however not without a connector, preferably at least 5 cm long, arranged between membrane and end side.

In certain exemplary embodiments according to the present invention, the membrane is flat or balloon-like (i.e. open at one end), but not tube-like (i.e. open at both ends).

In some exemplary embodiments according to the present invention, the membrane is, at least partially, designed as a sealing element, preferably at its edge, e.g. as a sealing ring. In these embodiments, for example in the region of the insertion opening, the membrane is arranged around the limb stump. It optionally prevents the fluid, which is present in the pressure chamber, from losing pressure along the limb stump. It may advantageously prevent an outflow of fluid into the exterior of the pressure chamber and thus prevent a pressure drop inside the pressure chamber.

In some exemplary embodiments according to the present invention, the membrane is, at least during use of the apparatus (i.e. when the limb stump of the patient is inserted in the interior of the pressure vessel) and at least in sections thereof arranged inside the pressure vessel. Preferably, it is present only and/or always in the interior of the pressure vessel. Alternatively or additionally, it is connected directly or indirectly in fluid-tight connection to sections of the wall of the pressure vessel.

In some exemplary embodiments according to the present invention, the membrane is also present exclusively inside the pressure vessel during use of the apparatus (i.e. when the limb stump of the patient is inserted in the interior of the pressure vessel).

In several exemplary embodiments according to the present invention, the membrane does not protrude from the interior of the pressure vessel, in particular not in the region of the second end side or in the region of the bottom area.

In some exemplary embodiments according to the present invention, the membrane is permanently connected to the apparatus. In certain embodiments according to the present invention, permanently means that the membrane cannot be detached from the pressure vessel without the use of tools or only destructively; for example, it can be permanently and yet releasably connected to the wall by a clamping ring or by clamping rings (not to be confused with the connectors in the sense of the present invention, which may additionally be provided) and one or more screws. A releasability using a tool may advantageously be provided in order to enable replacing the membrane, e.g., due to abrasion after a plurality of uses. In these embodiments, however, the membrane is not intended to be releasable from the pressure vessel by simple pulling over, pulling down or the like. At the same time, the permanent fastening may advantageously ensure that the forces which are transmitted in the pressure vessel by the fluid to the membrane during use of the apparatus cannot release the membrane from the pressure vessel or from its wall.

In some exemplary embodiments according to the present invention, the apparatus comprises no other axial reception for the free stump end than the membrane and/or no “axial reference compliant means”. The stump contacts preferably only the membrane. In other embodiments according to the present invention this may be different.

In some exemplary embodiments according to the present invention, the apparatus according to the present invention comprises no, in particular circular and/or disk-shaped cover of the insertion opening, which is made, e.g., of rubber and/or which is This is different in other embodiments; in such embodiments, the apparatus according to the present invention comprises at least one in particular circular and/or disk-shaped cover of the insertion opening, which is made, e.g., of rubber and/or which is optionally a single-piece with an integral central hole.

In some exemplary embodiments according to the present invention, the apparatus according to the present invention, comprises, in particular during its use, no sand, no plaster material, no curing material, in particular not in the pressure chamber or between wall and membrane.

In certain exemplary embodiments according to the present invention, the fluid is not sand, nor solid particles nor balls, in particular not polystyrene balls, or does not comprise suchlike.

In certain exemplary embodiments according to the present invention, the membrane is not made of, or comprises no, polyethylene.

In some exemplary embodiments according to the present invention, the apparatus according to the present invention, comprises no device, such as an elastic ring or a rubber ring, and in particular no rubber ring which is provided for fixing the membrane to an outer wall of the pressure vessel. In other embodiments according to the present invention this may be different. Such a rubber ring would not be a connection in the sense of the present invention.

In some exemplary embodiments according to the present invention, the apparatus according to the present invention comprises no vacuum source (in particular no vacuum source which is operated electrically or hydraulically) or is not connected to such in fluid communication.

In certain exemplary embodiments according to the present invention, the apparatus according to the present invention comprises no pressure source and/or no, in particular inflatable, expansion devices or other “expander means” or is not connected to such.

In some exemplary embodiments according to the present invention, the apparatus according to the present invention comprises no vacuum connection.

In certain exemplary embodiments according to the present invention, the apparatus according to the present invention comprises no air chamber.

In some exemplary embodiments according to the present invention, the apparatus according to the present invention comprises no feed screws.

In some exemplary embodiments according to the present invention, the apparatus is designed to manufacture a negative impression of the limb stump. The negative impression has a wall thickness of preferably 2 to 8 mm, particularly preferred 3 to 6 mm.

In some exemplary embodiments according to the present invention, the apparatus is not designed to manufacture a positive impression of the limb stump.

In some exemplary embodiments according to the present invention, the at least one connector is fixed to the bottom region, to the wall and/or to the second end side of the pressure vessel, preferably with or by one of its ends or end sections, whereas the other end or the other end sections is preferably connected to the membrane.

The connector may fix the membrane to the interior of the pressure vessel so that the membrane, in particular for the advantageously simple insertion of a limb stump into the interior of the pressure vessel, may not bulge or turn outward the insertion opening to the outside.

The connector may be fixed using mechanical fixing devices, e.g. hook(s), clamping connection, screws, rivets or the like, to the wall and/or to the second end side of the pressure vessel.

In several exemplary embodiments according to the present invention, the at least one connector is 10 long, in other embodiments at least 20 cm long, in further embodiments at least 30 cm long.

In several exemplary embodiments according to the present invention, the at least one connector does not serve for connecting the membrane in one region of the upper edge of the wall of the pressure vessel and/or not for establishing a fluid-tight connection between membrane and pressure vessel.

In some exemplary embodiments according to the present invention, the at least one connector is connected to a reinforced section of the membrane. A reinforced section of the membrane may comprise, for example, an increased wall thickness, other materials and/or material coatings

A reinforced section of the membrane may for example comprise higher strength. Increased forces, in particular frictional forces, on the reinforced section, for example when inserting a limb stump into the pressure vessel or when pulling the limb stump out of it, may, using the reinforced section of the membrane, be advantageously transferred via the connector.

Another section of the membrane which is not, at least not completely, exposed to these forces may comprise other material properties, for example in order to enable a more comfortable and softer seat of a limb stump in the membrane in the pressure chamber during the preparation of a plaster impression. In particular, the distal regions of a limb stump are often particularly sensitive to pressure and should, therefore, be relieved as much as possible during the preparation of a plaster impression.

In some exemplary embodiments according to the present invention, the reinforced section of the membrane is connected on one hand to the at least one connector and on the other hand to the wall or to the bottom of the pressure vessel.

In some exemplary embodiments according to the present invention, at least two connectors are connected at the circumference of the membrane. Likewise, three, four, five, six or more connectors may be connected at the circumference of the membrane. The connectors may be connected to the circumference of the membrane at a regular or irregular distance.

In certain exemplary embodiments according to the present invention, the pressure vessel comprises the insertion opening and optionally one or more inlets and/or outlets which, however, are all, or each, provided with one closure device for closing them in a fluid-tight manner. Otherwise, the wall of the pressure vessel or of the pressure chamber is fluid-tight in these embodiments.

In some exemplary embodiments according to the present invention, the inlets and/or outlets which are provided with a closure device are part of the wall

In certain exemplary embodiments according to the present invention, the pressure chamber is designed exclusively by the membrane and parts or sections of the wall, or exclusively by the membrane and parts or sections of the wall and fluid-tight connections between membrane and wall.

In some exemplary embodiments according to the present invention, an end side of the pressure vessel belongs to the wall, in particular the end side denoted herein as second end side.

In some exemplary embodiments according to the present invention, the pressure vessel is locked or lockable in a fluid-tight manner at its second end side.

The pressure vessel may comprise a first and a second end side. The insertion opening may be an opening on an end side. Preferably, it is situated on the first end side or in the region of the first end side.

The membrane may be connected releasably or non-releasably to the wall or to another section of the pressure vessel.

In some exemplary embodiments according to the present invention, the medical apparatus comprises at least one outlet which is, or enables, a fluid connection between the pressure chamber and the exterior of the pressure vessel. Further, it comprises a valve, a stopcock or other closure device for reversibly closing the outlet or the fluid connection.

The outlet may advantageously be used to lower the pressure present in the pressure chamber by discharging fluid from the latter. This may be necessary or helpful for inserting the limb stump or for adjusting an insertion depth of the stump.

In some exemplary embodiments according to the present invention, the pressure vessel comprises a first and a second end side. In this, the outlet of the pressure vessel is optionally arranged in the region of the second end side or in the region of an end side of the pressure vessel.

In some exemplary embodiments according to the present invention, the pressure vessel is connected to the pressure source via the outlet or with an inlet, which is separated therefrom. There may thus be two passages in the wall, namely, outlet and inlet.

In some exemplary embodiments according to the present invention, the pressure chamber of the pressure vessel comprises water or another fluid in its interior or it is filled therewith. Due to its lack of compressibility, a liquid leads to more reproducible results in the production of the plaster impression. Thereby, water is the cheapest and most-easily available variant.

In certain exemplary embodiments according to the present invention, the wall of the pressure vessel is transparent in at least sections thereof. This may allow visual monitoring of the insertion depth and other aspects during the manufacture of the plaster impression by the orthopedic technician in a simple manner.

In some exemplary embodiments according to the present invention, the pressure vessel comprises one or more markings in the region of its at least one transparent section. The insertion depth of the limb stump may be read based on said markings.

In some exemplary embodiments according to the present invention, the pressure vessel comprises a multi-part sealing ring, in particular an upper-thigh sealing ring (not to be confused with a connector in the sense of the present invention), wherein in other embodiments, it comprises no such ring. If said ring is provided, it may be detachably connected, for example screwed, to the pressure vessel in the region of the insertion opening or of a clamping ring or a fastening ring carrying the membrane, respectively. Said ring can prevent or limit the floating of the membrane into an exterior of the pressure vessel due to the pressure prevailing on the other side of the membrane. This may lead to more correct pressure conditions and results of the impression. In addition, the membrane may be protected from a shearing off, for example at the edge of the insertion opening or from another injury of the membrane, for instance in the gap between the limb stump and the edge of the insertion opening. One or several connectors in the sense of the present invention may be additionally provided

In some exemplary embodiments according to the present invention, the pressure chamber is formed or limited by at least a portion of the wall and by the membrane. The membrane is connected to the section of the wall, preferably in the region of the second end side of the pressure vessel, in a material connection, force-fit connection and/or form-fit connection. By the connector, an undesirable bulging, moving, floating or stretching of the membrane towards the top or into the exterior of the pressure vessel, in which the membrane—unlike in the interior of the pressure vessel—is not laterally supported by the wall, may in some embodiments according to the present invention be prevented or limited to an acceptable dimension. The connector thus holds the membrane, at least substantially, optionally inside the pressure vessel. The latter, or the connector, may counteract an undesirable floating of the limb stump. The floating may have an unfavorable influence on the pressure prevailing in the pressure chamber and acting on the limb stump through the membrane, in that the membrane no longer contacts the stump with uniform pressure in all sections in which it surrounds the stump. Therefore however, as there are no optimal pressures at work at the stump, this results in that the produced plaster impression has not been produced under subsequent loads, which will occur when walking with the prosthesis to be produced. Furthermore, reduced or prevented floating may contribute to protecting the membrane which is protected against damage in the interior of the pressure vessel by the wall of the latter. When providing the connector according to the present invention, there is advantageously no need for any other limitation of the floating of the membrane, e.g. by a ring which engages tightly on the thigh. Since such a ring would have to be provided in a plurality of sizes in order to be able to manufacture plaster models with the apparatus according to the present invention for a plurality of differently thick limb stumps, the skilled person is offered a simplicity that is easy to understand. In addition to simplifying the use of the apparatus, it also means saving in material for rings, costs and the like.

In some exemplary embodiments according to the present invention, the connector is connected to the membrane in a section of the membrane which lies in the region of the insertion opening.

In certain exemplary embodiments according to the present invention, the membrane is connected to the pressure vessel in the region of the insertion opening or of the first end side of the pressure vessel, and additionally in a second region or section of the pressure vessel which is different therefrom. The second region may preferably be in the interior of the pressure vessel and/or in the pressure chamber. The membrane may preferably be connected to the second region by the connector. The membrane may preferably be in contact only indirectly with the second region, namely optionally by the connector, i.e. preferably not having, itself, a direct contact with the second region, therefore, preferably not touching the latter.

In some exemplary embodiments according to the present invention, the second region is an edge section of the bottom region or of the second end surface or end side

In some exemplary embodiments according to the present invention, the connector is an elastic spring or comprises an elastic element.

In some exemplary embodiments according to the present invention, the connector is not elastic nor stretchable.

In some exemplary embodiments according to the present invention, the connector is length-adjustable. Said length may be adjusted by a corresponding adjusting device, which may preferably be adjusted from outside the pressure vessel. As a result, the distance between the distal end of the membrane and, for example, the bottom region, the lower end side or end surface of the pressure vessel is changed. This allows to adjustably arrange the membrane within the pressure vessel which in turn may allow an optimal adjustment of the apparatus according to the present invention with regard to the specific limb stump regardless of its length.

In some exemplary embodiments according to the present invention, the connector directly or indirectly connects the membrane to the end side of the pressure vessel which lies opposite to the end of the pressure vessel comprising the insertion opening, i.e. connecting it in particular to a lower end side, end surface or bottom region.

In some exemplary embodiments according to the present invention, the connector does not connect the membrane to a middle or central region of the end surface, end side or bottom region. This allows or promotes a comparatively free arrangement of the limb stump inserted in the membrane within the pressure vessel, without the connector applying pressure on distal sections of the limb stump through the membrane.

In some exemplary embodiments according to the present invention, connecting is to be understood as form-fit connection and/or force-fit and/or material connection.

In some exemplary embodiments according to the present invention, the connector is arranged to connect a during-use upper section of the membrane.

In some exemplary embodiments according to the present invention, the connector is arranged to keep a space or distance within specified limits between the section of the membrane connected to the connector on one side and the section of the bottom region/lower end side/end surface, likewise connected to the connector, on the other side. The distance may be, e.g., constant when using a non-elastic connector.

In some exemplary embodiments according to the present invention, the connector is directly connected to the wall. The connector may hereby be the result of a joining process e.g. an adhesive strip, a rivet or the like.

In some exemplary embodiments according to the present invention, the membrane touches, in the region of its connection, the bottom region, bottom side or lower end side; in other embodiments the membrane or material thereof does not touch the aforementioned.

In certain exemplary embodiments according to the present invention, the membrane is made of or comprises material which comprises in a first direction of the material another elasticity or extensibility than in a second direction which is different from the first direction, wherein the second direction may optionally be perpendicular to the first direction. In these or in other exemplary embodiments according to the present invention, the specific elasticity or extensibility in the first and/or in the second direction, respectively, is achieved by fibers, which the membrane comprises, which are e.g. embedded in silicone, a silicone matrix or another material, preferably a fluid-tight material. Such fibers may extend in the first and/or second direction of the membrane, substantially extend in the first and/or second direction and/or substantially work in the first and/or second direction. In these or in other embodiments according to the present invention the different elasticities or extensibilities may be additionally or alternatively achieved by other designs.

The first direction may, when the membrane is ready for use or is fixed as intended on the pressure vessel, be an insertion direction of the limb stump or a longitudinal direction of the pressure vessel. The second direction may be in the angle, e.g. in the right angle, relative to the first direction.

Fibers which extend or run or work in the first direction are optionally not stretchable; or are stretchable only to a limited extent. Optionally, they are less stretchable than optionally provided fibers which extend or run or work in the second direction. Fibers which run, extend or work in the second direction may, if present, be stretchable, they may be more stretchable or more elastic than the fibers of the first direction; their extensibility may optionally correspond to that of the fibers which extend in the first direction. The fibers extending in the first direction are optional, so are the fibers extending in the second direction. The extensibility or elasticity of the fibers may correspond or correlate to that of the membrane. The fibers may be made of, or comprise, nylon. They may be made of tension-proof material, in particular of material which is more tension-proof than the material embedding it. Such a design, which e.g. prevents, or significantly limits, a longitudinal stretching (which may optionally be the first direction), allowing at the same time a stretching of the membrane in the circumferential or radial direction for adapting it to the limb stump, may also advantageously counteract an undesired floating of the limb stump. In addition, the relationship between the depth of the pressure chamber and the insertion depth within the membrane may advantageously remain the same. Moreover, the above-mentioned multi-part sealing ring or upper-thigh sealing ring may be omitted. According to the present invention, it may further be possible to produce plaster impressions by using only one membrane, at a whole row of limb stumps which are differently thick in the cross section. The membrane advantageously fits sufficiently tightly on thick as well as on thin stockings in order to prevent the creasing of the membrane described herein. The cross-section or circumferential extensibility which the membrane may comprise in some embodiments according to the present invention even when the longitudinal extensibility is prevented or limited, advantageously contributes thereto.

In some exemplary embodiments according to the present invention, the membrane is partially, in at least one section thereof or completely or as a whole less than 2 mm thick, preferably less than 1 mm. With this thickness, it distorts the difference between the actual geometry of the limb stump and the plaster in a negligible manner, at most.

In certain exemplary embodiments according to the present invention, the pressure vessel comprises a closable air release opening with a corresponding closure device. Air can escape from it, in its non-closed state, out of the pressure chamber. This is advantageous when filling the pressure chamber with liquid for the first time; the apparatus, if it is intended to be used filled with liquid, does not have to be purchased or delivered filled with liquid. It can be air-filled and therefore be remarkably lighter for transportation. The air present may be released out of the closable air release opening when filling with liquid.

In some exemplary embodiments according to the present invention, the medical apparatus comprises a device by means of which at least the pressure vessel is vertically adjustable relative to an underground on which the apparatus rests or on which the patient is standing with his healthy leg.

In certain exemplary embodiments according to the present invention, the wall of the pressure vessel is made of plastic, for example Plexiglas or polycarbonate, or comprises one or more of these materials.

In certain exemplary embodiments according to the present invention, the apparatus does not comprise an edge or ring with an opening for the patient's thigh, which edge or ring is designed to be releasably connected to the pressure vessel and/or the wall.

In certain exemplary embodiments according to the present invention, the pressure vessel and/or the wall comprise no air opening.

In certain exemplary embodiments according to the present invention, the proximal section of the membrane corresponds to the upper section of the membrane during use of the apparatus.

The proximal section may include the entire upper half.

It may include the upper 0% to 30% or up to 40% of the membrane.

A proximal or upper section of the membrane may extend from the edge of the membrane, being free or connected to the pressure vessel, which the patient is passing with the limb stump when entering the membrane during use of the apparatus according to the present invention, along the first 10, 20, 30 reaching up to 40 to 50% of the entire length of the membrane.

The entire length may optionally be determined from the height at which the membrane is clamped or screwed or differently connected

The entire length may optionally be determined from the insertion opening of the pressure vessel up to the distal end of the membrane.

In certain exemplary embodiments according to the present invention, the connector, the membrane and/or the bottom region comprises at least one thread for directly or indirectly screwing the membrane or a component connected therewith to the pressure vessel.

In certain exemplary embodiments according to the present invention, the connector limits a floating of the membrane caused by fluid in the pressure chamber such that a contact point surface between limb stump and membrane is at the same height level as a transition section, in which section the pressure chamber is not formed by the wall (or by a component, e.g. a clamping ring, connected thereto) anymore rather by the membrane and/or the membrane is not supported anymore, in particular not radially or laterally or at its sides, by the wall (or by a component, e.g. a clamping ring, connected thereto).

In certain exemplary embodiments according to the present invention, the membrane is made of or comprises a material which comprises, in a first direction and/or in a second direction thereof, fibers embedded in a matrix or otherwise connected thereto.

In certain exemplary embodiments according to the present invention, the membrane, which optionally comprises a matrix, or its matrix is made of, or comprises, silicone.

In certain exemplary embodiments according to the present invention, some or all of the fibers have a wavy, curvy or zig-zag pattern.

In certain exemplary embodiments according to the present invention, the membrane is non-extendable or non-elastic in a first and/or second direction thereof.

In certain exemplary embodiments according to the present invention, “non-extendable” or “non-elastic” means that the modulus of elasticity of the respective component (connector, membrane, fibers, etc.) is at least above 700 N/mm², preferably above 1000 N/mm², especially preferably above 2000 N/mm².

In certain exemplary embodiments according to the present invention, non-extendable or non-elastic means that an extensibility of the respective component (connector, membrane, fibers, etc.) is not more than 20%, preferably not more than 10%, preferably not more than 5%, particularly preferably not more than 2% of its length before the component tears or breaks

In certain exemplary embodiments according to the present invention, the membrane, fibers thereof and/or the connector has a modulus of elasticity such as nylon.

In certain exemplary embodiment according to the present invention, in addition to the upper and/or the proximal section of the membrane and/or in addition to the section of the membrane which lies in the region facing the insertion opening of the pressure vessel, also a lower and/or distal section of the membrane and/or a section which lies in the region of the membrane facing away from the insertion opening of the pressure vessel and/or facing a bottom region is preferably releasably, directly or indirectly, fixed at or to a section of a wall of the fluid container, e.g. a bottom region or side region thereof, by a connector referred to herein as a distal connector.

The same that applies herein to connectors, in particular with regard to their material, length, elasticity, extendibility and other properties, in particular material property, also applies to the connector referred to herein as a distal connector, provided that this does not create any recognizable contradiction in the view of the person skilled in the art.

The distal connector may be configured and/or provided in any embodiments according to the present invention, as in the WO 2016/135320 A1, the disclosure of which is hereby also fully incorporated herein by reference in its entirety as subject matter of the present application.

In some exemplary embodiments according to the present invention, the connector and/or the distal connection are present in the interior of the pressure vessel completely and without contact to the exterior of said pressure vessel.

In some exemplary embodiments according to the present invention, the pressure vessel and/or line communicating herewith comprise a pressure gauge which determines or measures the pressure prevailing in the pressure chamber. The orthopedic technician may, for example, orientate himself according to the measured pressure for the a.m. pressure increase.

In some exemplary embodiments according to the present invention, the membrane is deformable to the inside or to the outside, preferably elastically extensible. In others, it is elastic in one direction but it is not elastic or comparatively or substantially less elastic in a direction which is particularly perpendicular thereto.

In some exemplary embodiments according to the present invention, the membrane is arranged such that it can be pushed during filling the pressure chamber with fluid by this fluid that fills the pressure chamber, through the insertion opening into an exterior of the pressure vessel.

In certain exemplary embodiments according to the present invention, the membrane is extensible, however it remains closed in its extended state, except for one opening, similar to a finger of a rubber glove or an air balloon in the stretched state.

In some exemplary embodiments according to the present invention, the membrane is arranged on the pressure vessel in order to seal it on the end side. This preferably takes place by not using the connector.

In some exemplary embodiments according to the present invention, the apparatus has no opening of a connecting tube in an annular or cylindrical space between the, preferably elastically stretchable membrane and the wall of the pressure vessel.

In some exemplary embodiments according to the present invention, the apparatus comprises a fastening ring or stump seal ring which, for example, comprises or consists of at least two half-shells or part-shells (in short: shells). The fastening ring may be placed on the end side of the pressure vessel (and, for example, may be connected thereto by suitable connecting elements such as, purely exemplarily, screws), by limiting the insertion opening for the stump. The fastening ring may optionally or preferably prevent a floating of the membrane during the use of the apparatus. Such a fastening ring or stump seal ring is no connector according to the present invention. Such a ring may be provided in addition to such a connector.

In some exemplary embodiments according to the present invention, the apparatus comprises no carriage on which the pressure vessel would be mounted.

In some exemplary embodiments according to the present invention, the apparatus comprises no electrically and/or hydraulically or hydraulically driven device for generating pressure in particular arranged for generating pressure on the membrane, or is not connected to such a device.

The apparatus according to the present invention may be equipped with one or more features in any combination as disclosed in WO 2016/135320 A1, the disclosure of which is hereby fully incorporated by reference also as the subject-matter of the present application

In some exemplary embodiments according to the present invention, the membrane is not tubular, i.e. not open at both ends thereof.

In certain exemplary embodiments according to the present invention, the membrane is made of fluid-tight, in particular water-tight, material. It can be made of or comprise silicone. It can be made of or comprise fiber-reinforced silicone. A co-polymer or a rubber may be provided instead of silicone.

Some or all of the embodiments according to the present invention may comprise one or several of the advantages mentioned supra or in the following.

It is obvious that the prosthesis shaft is useful to the patient due to the regained mobility, especially when standing and walking. When standing and walking, when the shaft is loaded according to its intended purpose, the shaft must therefore sit especially comfortably. The methods known so far for producing the plaster impression or for measuring the dimensions of the stump do not adequately comply with or meet or fulfill this, since they do not take into account the soft tissue displacement in the stump, e.g. relative to the bony portion of the stump, as they occur later when the shaft is subjected to stress, due to the lack of loading the stump during measuring or plastering. The result may lead to an inaccuracy in the manufacturing of the shaft which, even when determining the body dimensions at precisely defined heights and regions, lies in the cm-range. This is advantageously not the case when the apparatus according to the present invention is used while the patient is standing; the stump undergoes almost identical loads and soft tissue displacement as in the later load in the shaft.

By the present invention, it is advantageously possible for the first time with the patient standing and loading the stump during the manufacturing of the plaster impression or the measuring to reliably and above all reproducibly produce a plaster impression of the patient's stump or to measure the dimensions of the stump, in order to obtain a data model which reflects or mirrors the stump.

Thus, the present invention allows to make a plaster impression or to obtain a stump model as a basis for a shaft that is conveniently fitting the person wearing it, especially when walking and standing. Less skill is needed than hitherto required.

Thus, the present invention enables the manufacturing of shafts for prostheses of human upper and lower extremities in an objective manner based on directly obtained measurements data. The present invention thus enables the production of a well-adapted prosthesis shaft, wherein however the expensive activities which are executed purely subjectively and manually and which are required in the methods of the state of the art, may be avoided.

By the present invention, during the manufacturing of the plaster impression of a standing patient and thereby having an almost realistically loaded stump, it is possible for the first time already during the manufacturing of the plaster impression or during the measurements to obtain a feedback from the patient concerning tight or painful regions or points. Using the apparatus according to the present invention, the condition or situation of the shaft when worn later is so to say already felt “in advance” by the patient during the production of the plaster impression or during the measurements; unsatisfactorily fitting sections of the later shaft are recognized earl or anticipated. In this way, desired changes or upholstering may be recorded and prepared by the orthopedic technician already during the manufacturing of the plaster impression and the measuring of the patient. This may, over the time which passes until the final, fitting shaft is present, (help) saving significant amount of work and time.

Since the patient is able to use the apparatus while standing and thereby loading the membrane with parts of his body weight, there is no need for precautionary measures to protect the limb stump from being displaced by axial reaction forces which may act on the limb stump by the pressure chamber and may push or displace the limb stump out of the interior of the pressure vessel. In other words, there is no need for the patient to be held in position—relative to the pressure vessel. His body weight may fulfill this.

Attaching or connecting the connector to a proximal or upper region of the membrane advantageously avoids radial pressure on the membrane in sections lying further distal thereto. This allows pressure, which could cause or effect radial compression to or on the limb stump inserted in the membrane, to be avoided in an anatomically weaker section of the limb stump as compared to more proximal sections. As a result, a more desirable geometry of the generated plaster impression may be achieved, above all in the distal stump section. Attaching the connector at a proximal section of the membrane avoids such deformation of the distal part of the limb stump, which may advantageously result in a correct geometry of the plaster impression.

The present invention allows, in contrast to the conventional apparatuses of the state of the art, to produce shafts for prostheses in an objective manner. This ensures a better supply thanks to improved fitting and may reduce the production costs by requiring no or only a minor expensive manual processing and adaptation. In addition, the patient care can be accelerated since the time-consuming adaptation steps may, at least in number, be greatly reduced, or even be completely eliminated.

The present invention is exemplarily explained with regard to the accompanying drawings in which identical reference numerals refer to the same or similar components. The following applies in the, partly, highly simplified figures.

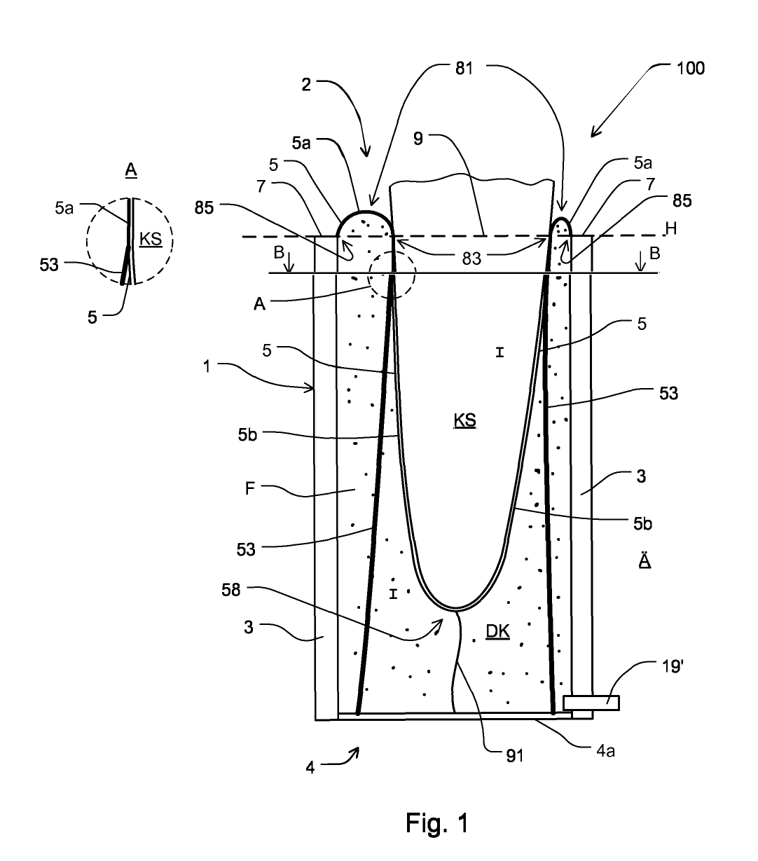

FIG. 1 shows a longitudinal cut of a first embodiment of an apparatus according to the present invention, from the side;

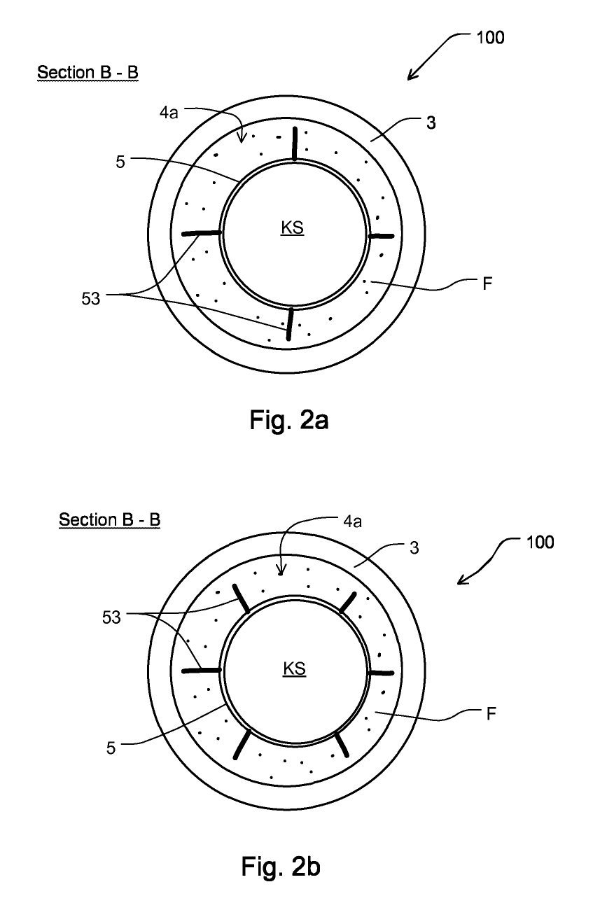

FIG. 2a, b show the apparatus according to the present invention of FIG. 1 in several embodiments in a further sectioned plane; and

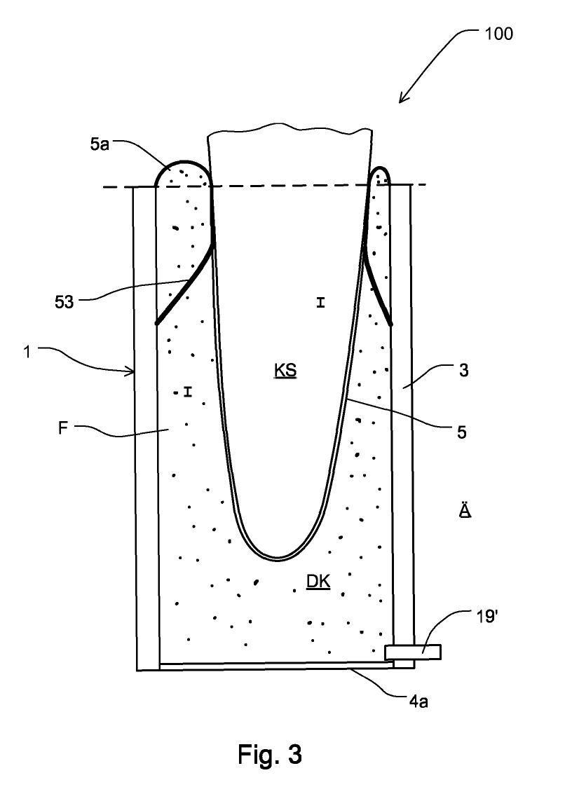

FIG. 3 shows a longitudinal cut of an apparatus according to the present invention in a further embodiment, from the side.

FIG. 1 shows a longitudinal cut (i.e. with respect to FIG. 1 from top to bottom) of a first embodiment according to the present invention of an apparatus 100. When herein “distal” is mentioned, it means a position at or direction to the bottom. “Proximal” is correspondingly on top, higher or in direction to the top.

The first embodiment shows a pressure vessel 1 with a pressure chamber DK, a wall 3 and a membrane 5. The membrane 5 comprises a first or proximal membrane section 5a, which is connected to an upper edge 7 of the wall 3. The first membrane section 5a is optionally reinforced in this embodiment, shown by a thicker wall thickness (shown in FIG. 1 by a thicker line width). A reinforced embodiment may alternatively or additionally be done or achieved by a different choice of material, a fiber-reinforced composite material, a material coating or in another way.

Furthermore, the membrane 5 comprises a second or distal membrane section 5b, which is arranged in the middle section of the membrane 5 and is connected to the first membrane section 5a or is integral therewith. The second membrane section 5b is optionally not reinforced, which is represented by a smaller wall thickness (indicated in FIG. 1 by a thinner line thickness).

The two different membrane sections 5a, 5b serve different load situations of the membrane 5. When inserting and withdrawing a limb stump KS into the membrane 5, and thus into the pressure vessel 1, particularly the first membrane section 5a is loaded with frictional force between the limb stump KS and the membrane 5. Due to this increased load, the first membrane section 5a is optionally made reinforced. The second membrane section 5b has in particular a sealing function and holding function of the limb stump KS in the pressure vessel DK with lower load compared to the first membrane section 5a.

Two connectors 53, the function of which will be described in more details below, are optionally arranged in this exemplary embodiment at the connection or interface between the first membrane section 5a and second membrane section 5b or in the transitional region between the two respectively. In particular, the connectors 53 are connected to the first membrane section 5a (optionally alternatively at a point or position other than the one shown in FIG. 1).

The connection between connectors 53 and membrane section 5a or 5b is designated by the reference “A”. In the example of FIG. 1, the connection A lies at the distance B below the height H and/or the upper edge of the wall 3.

The distance B may optionally be between 0 cm and 40 cm, preferably between 5 cm and 30 cm, particularly preferably between 8 cm and 10 cm.

The region of the connection A is shown enlarged on the left in FIG. 1.

The membrane 5 is preferably releasably fastened to or at the bottom region 4a, using the at least two connectors 53, which extend from a proximal section of the membrane 5, alternatively, to a region of the wall 3 (for instance in its upper, middle or lower region).

The proximal region of the membrane 5 is to be understood herein as the upper third of the membrane 5 or as a region in the upper third of the membrane 5, by way of example

The proximal region of the membrane 5 is recognizable facing the insertion opening 9 or is located in the vicinity thereof.

The region with which the membrane 5 is connected to the connector 53 is not located in a central, distal section 58 of the membrane. This advantageously prevents radial pressure onto the membrane 5 in a distal section due to the connector preventing the floating of the limb stump KS. This pressure could effect a radial compression of the limb stump KS stuck in the membrane 5 which may lead to an undesirable geometry of the generated plaster impression above all in the distal stump section. The provision of the connector 53 in a proximal section may help to avoid such deformations of the distal part of the limb stump This may advantageously result in a correct geometry of the plaster impression. Although the connector 53 effects a radial pressure onto the limp stump KS in a proximal section, due to its proximal connection to the membrane. However, there (i.e. proximal or with regard to FIG. 1 “at the top”) the limb stump KS comprises a stronger anatomy and is less susceptible to radial compression which is avoidable by the present invention in some embodiments.

The pressure vessel 1, shown in FIG. 1 as purely optionally cylindrical, comprises optionally a first end side 2 (at the top in FIG. 1) and a second end side 4 (at the bottom in FIG. 1). The second end side 4 in the exemplary embodiment of FIG. 1 is fluid-tight sealed with a bottom plate or bottom region 4a against an exterior Ä. The bottom region 4a may be made of the same material as the wall 3. It may be manufactured in one piece therewith

The membrane 5 separates, in a fluid-tight manner, the fluid container or pressure chamber DK of the pressure vessel 1 from an exterior of the fluid container or pressure chamber DK, or exemplarily from the exterior Ä, i.e. a surrounding of the pressure vessel 1, or, as shown in FIG. 1, against a limb stump KS inserted into, or surrounded by, the membrane 5.

The membrane 5 may be fluid-tight connected to the pressure vessel 1 at an upper, often circular, rectangular, square or differently shaped circumferential edge 7 of the wall 3, or at another site.

The upper edge or rim 7 is exemplarily situated in a plane in which there is an insertion opening 9 of the pressure vessel 1 or it delimits said insertion opening 9 at its circumference. The insertion opening 9 is optionally situated in the plane H which is indicated with a dashed line.

The insertion opening 9 serves inserting the limb stump KS which is wrapped with a wet plaster bandage into an interior I of the pressure vessel 1.

The interior I is the volume delimited by the wall 3 of the pressure vessel 1. It extends from the second end side 4, which is fluid-tightly sealed with the bottom region 4a, to the insertion opening denoted with 9 and indicated by a dashed line.

The pressure chamber DK is filled with a fluid, here exemplarily with liquid F indicated with points. A filling with gas is also contemplated or covered by the present invention.

In FIG. 1, the apparatus 100 is illustrated in a state in which the extremely schematically-indicated limb stump KS of the standing patient is inserted into the interior I such that it is surrounded by the membrane 5 at least in its distal section. The membrane 5 contacts the plaster bandage covering the limb stump KS like a second skin, wherein there may be further layers, like liners or the like, provided between the plaster bandage and the membrane 5.

The limb stump KS is in the example of FIG. 1 loaded with the full body weight of the standing patient. The amount of the liquid F is measured with regard to the known volume of the interior I or the pressure vessel 1 such that the limb stump KS may enter through the insertion opening 9 into the pressure vessel 1 at least so deep or far that the entire region of the plaster bandage contacts the membrane 5; at least as much as it is relevant for the plaster impression. At the same time, the amount of liquid F is measured such that the distal end of the limb stump KS (at the bottom in FIG. 1) does not touch the bottom of the pressure vessel 1 or does not support itself on the bottom. In this way it is ensured that the patient rests with the inserted extremity on the pressure of the fluid and that the plaster bandage is subject to the same pressure at each point by the membrane 5.

As is shown in FIG. 1, the membrane 5, when no limb stump KS is inserted into the pressure vessel 1, lifts up or floats due to the pressure of the fluid, here of the liquid, and a liquid level appears (not shown in FIG. 1). Hence, the shape of the membrane 5, which is shown in FIG. 1, represents the form which the membrane 5 adopts under loading when it fits closely to the inserted limb stump KS and is pushed under elastic stretching by the latter —in the example of FIG. 1—into the depth of interior I towards the bottom region 4a

It is further to be seen in FIG. 1 that due to the fact that both the wall 3 and the membrane 5 prevent a fluid exchange between the pressure chamber DK and the exterior

A or a fluid leakage from the pressure chamber DK, they, hence, allow that the desired pressure builds up within the pressure chamber DK of the pressure vessel 1. However, they do not permit it to escape from the latter or to dissipate.

As is seen in FIG. 1, the pressure chamber DK is thus optionally formed by the membrane 5 and at least by parts of the wall 3 which, in this example, include also the bottom region 4a of the end side 4.

In exemplary embodiments of the present invention unlike those shown in FIG. 1, the pressure chamber DK may consist of or comprise a completely closed membrane, which may lie in the interior I of the pressure vessel 1 like a balloon or a bubble.

Furthermore, FIG. 1 shows all optional outlet 19′ in the side wall or wall 3 of the pressure vessel 1. If provided, this outlet may also be positioned elsewhere at the pressure vessel 1, e.g. in its bottom area.

The outlet 19′ connects the interior I in the region of the pressure chamber DK to the exterior Ä of the pressure vessel 1. It allows the filling level and/or pressure within the pressure chamber DK to be specifically modified, for example by discharging fluid from the pressure chamber DK via the outlet 19′. For this purpose, the outlet 19′ has a valve or stopcock (not presented in detail) which can be fluid-tight closed. Regardless of its designation as an outlet, the latter may also be used for introducing fluid and thus for the filling of the pressure chamber DK.

The membrane 5 is in the embodiment of FIG. 1 purely exemplarily connected to the bottom region 4a by the connectors 53 or the connector 53. The connector 53 is not (or only slightly) elastic or stretchable. The membrane 5 is optionally also not (or only slightly) elastic or stretchable in the longitudinal direction.

The connector 53 allows the membrane 5 to float proximally (upward in FIG. 1) to a small and/or predetermined extent. Therefore, a more or less annular, but in any case, closed in its circumference, uplifting or floating 81 arises at the second end side 2. The uplifting or floating 81 is to be seen, due to the sectional illustration in FIG. 1, as protrusions left and right of the limb stump KS. The connector 53 may be provided in order to prevent such a floating

The membrane 5 fits the limb stump KS (or a plaster bandage, a liner or the like being pulled thereover) closely at a contact point surface 83, which is more or less annular but, in any case, closed in its circumference. The contact point surface 83 is the circumferentially closed line or surface which comprises the points at which the limb stump KS has a final or closing proximal contact with the membrane 5. The contact point surface may also be referred to as contact region or contact line

At the same time, the membrane 5, at its radial circumference in the region of the second end side 2 of the wall 3, contacts, or is fixed with, a transition section 85 which is likewise closed in its circumference. The transition section 85 may be understood to be a region in which the limitation of the pressure chamber DK passes or merges from a limitation by the wall 3 into a limitation by the membrane 5. This can be well seen in FIG. 1 in that the pressure chamber DK below the arrow tip of the reference numeral line of the reference numeral 85 is only limited by the wall 3; above only by the membrane 5, which cannot support itself against a section of the wall 3

The fixing of the membrane 5 in the transition section 85 may be done in many ways. However, in this and in arbitrary other embodiments, it is not fixed by the connector 53. The fixing of the membrane 5 by the connector 53 takes place in the area of the connection A.

The floating 81 may be adjusted by either releasing or introducing fluid through the outlet or inlet 19′ in interaction with the connector 53 such that the contact point surface 83 and the transition section 85 are at the same height H (H is the height above the bottom region 4a) indicated by the dashed line in FIG. 1. Based on the experience of the inventor of the present invention, once this happens, optimal pressure conditions for manufacturing the plaster impression are prevailing.

Even if the connector is not adjustable in its length, it advantageously ensures that a predetermined extent of floating 81 beyond the height H cannot occur. A predetermined extent may be 1 to 4 cm, more preferably about 2 cm.

FIG. 1 shows a distal connector 91 which may be optionally provided—in any arbitrary embodiment—although it is not shown in FIG. 3.

FIG. 2a shows the apparatus 100 according to the present invention of FIG. 1 in a further sectional plane B-B. The sectional plane B-B shows the apparatus 100 in cross section at a position in the longitudinal direction in which the connectors 53 are connected to the membrane 5. The view direction of the sectional plane B-B is directed in FIG. 2a downwards to the bottom region 4a. In FIG. 2a four connectors 53 are arranged each being offset by approximately 90 degrees in the circumferential direction. Alternatively, the connectors could be arranged irregularly about the circumference. They may also be placed in any number and at any distance from each other (about every 5 cm along the circumference).

The connectors 53 are fastened at one of their ends to the membrane 5, and on their other end they are fastened to the bottom region 4a. The limb stump KS is arranged in the center of the membrane 5.

The connectors 53 may be connected to each other, for instance in that they, for example all of them, radiate in an annular device or are connected to an annular device which is guided respectively around the circumference of the membrane 5.

FIG. 2b shows the arrangement of FIG. 2a, however with six connectors 53, which are each offset by approximately 60 degrees in the circumferential direction. Alternatively, the connectors could be arranged irregularly around the circumference.

FIG. 3 shows the longitudinally cut apparatus 100 in a further embodiment from the side. In this embodiment, the connectors 53 are fastened to the wall 3 of the pressure vessel 1. Functionally, the connectors 53 fastened to the wall have the same task, namely to prevent unwanted floating of the membrane 5. A fastening to the wall 3 may have advantages in terms of assembly and/or handling during use of the apparatus 100 according to the present invention.

### LIST OF REFERENCE NUMERALS

100 apparatus
1 pressure vessel
2 first end side
3 wall
4 second end side, may optionally be closed or sealed by the bottom region
4a bottom region
5 membrane
7 upper rim or edge
5a proximal or first membrane section; reinforced section of the membrane
5b distal or second membrane section
9 insertion opening
19′ outlet, inlet
53 connector
58 central or middle region or section of the membrane
81 uplifting or floating of the membrane
83 contact point surface or plane
85 transition section
91 distal connector
A connection
Ä exterior of the pressure vessel
B distance
DK pressure chamber of the pressure vessel
I interior of the pressure vessel
F fluid or liquid
H height of the pressure chamber
KS limb stump

1. A medical apparatus for use in the preparation of a plaster impression or a data model of a limb stump, in particular of a lower leg stump, wherein the apparatus comprises at least:
a pressure vessel with a fluid container or a pressure chamber for receiving or storing a fluid, in particular one being under pressure, wherein the pressure vessel comprises a wall made of a first material, wherein the wall limits an interior of the pressure vessel against an exterior, wherein the pressure vessel comprises an insertion opening for inserting the limb stump into the interior of the pressure vessel; and
a fluid-impermeable membrane made of a second material, the membrane being arranged to form or limit the fluid container or the pressure chamber,
wherein at least one section of the membrane is fastened to the pressure vessel via at least one connector,
wherein the section lies in an upper region, in a proximal region or in a region facing the insertion opening.

2. The medical apparatus according to claim 1, wherein the at least one connector is fastened to a bottom region, to at least one of the wall and the second end side of the pressure vessel.

3. The medical apparatus according to claim 1, wherein the at least one connector is connected to a reinforced section of the membrane (5).

4. The medical apparatus according to claim 3, wherein the reinforced section of the membrane is both connected to the connector and to the wall of the pressure vessel.

5. The medical apparatus according to claim 4, wherein at least two connectors are fastened to a circumference of the membrane.

6. The medical apparatus according to claim 1, further comprising:
an outlet which is a fluid communication between the fluid container or pressure chamber and the exterior of the pressure vessel; and
a valve, a stop or lock device or a stopcock for opening and closing the outlet or the fluid communication.

7. The medical apparatus according to claim 1, wherein the connector is at least one of not elastic and not stretchable.

8. The medical apparatus according to claim 1, wherein the membrane (5) comprises a material having another elasticity or stretchability in a first direction of the material than in a second direction, the second direction being optionally perpendicular to the first direction.

9. The medical apparatus according to claim 1, wherein the membrane comprises a material which comprises, in at least one of a first direction and a second direction of the material, fibers which are embedded into a matrix or which are preferably connected to a matrix in a different way.

10. The medical apparatus according to claim 1, wherein the membrane is not stretchable or is not elastic in at least one of a first direction and a second direction thereof.

11. The medical apparatus according to claim 1, wherein the at least one section of the membrane is releasably fastened to the pressure vessel via the at least one connector.

12. The medical apparatus according to claim 3, wherein the reinforced section of the membrane preferably comprises a thicker wall thickness compared to the non-reinforced section of the membrane and/or preferably comprises a third material.

13. The medical apparatus according to claim 3, wherein the reinforced section of the membrane preferably comprises a third material.
